(19) United States
(12) Patent Application Publication
(10) Pub. No.: US 2014/0207087 A1
(43) Pub. Date: Jul. 24, 2014

(54) Sunless Spray Tanning Devices and Methods

(71) Applicant: NORVELL SKIN SOLUTIONS LLC, Alexandria, TN (US)

(72) Inventor: Nathan Marling, Alexandria, TN (US)

(73) Assignee: NORVELL SKIN SOLUTIONS LLC, Alexandria, TN (US)

(21) Appl. No.: 14/059,198

(22) Filed: Oct. 21, 2013

Related U.S. Application Data
(60) Provisional application No. 61/716,519, filed on Oct. 20, 2012.

(51) Int. Cl. A61M 35/00

(52) U.S. Cl. CPC B05B 15/12; A61M 35/00
USPC 604/289

(57) ABSTRACT

A sunless spray tanning device includes at least one peristaltic pump coupled to a spray gun. A liquid reservoir for housing a sunless tanning composition may be coupled to the inlet of the peristaltic pump. The composition is forced via the peristaltic pump to a spray nozzle located on the spray gun for application onto the skin of a customer. The spray gun includes a high volume, low pressure (HVLP) spray gun in some embodiments. A solenoid valve is disposed between the peristaltic pump and the spray gun in some embodiments. The spray gun may be mounted on a tanning booth apparatus or alternatively may include a handheld spray gun device for manual spray applications in various embodiments.

### CROSS-REFERENCES TO RELATED APPLICATIONS

This application claims benefit of and domestic priority to U.S. Provisional Patent Application No. 61/716,519 filed Oct. 20, 2012, all of which is hereby incorporated by reference in its entirety.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND

The present invention relates generally to devices and methods for emitting sprays and more particularly to devices and methods for generating a spray of a sunless tanning composition.

Conventional spray tanning systems are generally known in the art to include a liquid spray tanning composition reservoir and a nozzle assembly coupled to the reservoir for emitting the spray tanning composition toward a human user's skin for applying the composition to the skin. A pump or compressor is also typically used to force the composition from the reservoir to the nozzle assembly. Spray tanning devices may take many forms. One common spray tanning device includes a handheld manual spray gun that an operator may use to apply spray tanning composition onto a customer's skin. Such spray guns often include a high volume, low pressure (HVLP) nozzle and pump configuration to achieve desired spray characteristics. Conventional handheld spray gun devices for spray tanning composition application often suffer from problems associated with inconsistent spray uniformity. These conventional devices typically use centrifugal or piston driven pumps for forcing spray from a reservoir to the spray nozzle assembly. Such conventional pumps are prone to problems such as clogging or accelerated wear and must be replaced frequently.

A second type of spray tanning device includes an automated spray booth wherein one or more spray nozzle assemblies are mounted on a fixed support, or alternatively on a moveable track, adjacent a full or partial enclosure. A customer typically stands in the enclosure, and the nozzle assemblies emit spray tanning composition toward the customer while the customer is standing in the enclosure. In these types of automated configurations, the nozzle assembly is typically not manually operated by a user, but is rather positioned by a motor-driven linkage system. A reservoir for storing spray tanning composition may be located on the enclosure or may be located remote from the enclosure. The spray nozzle assembly in such auto-booth devices may also include a conventional centrifugal or piston driven pump. As such, conventional auto-booth devices also suffer from many of the same problems experienced with manual HVLP spray gun systems, including clogging, problems with spray uniformity, and accelerated wear. In some applications, conventional spray tanning devices using piston-driven pumps cause an undesirable pulsating spray that emits less spray during the top portion of the piston stroke and delivers more spray during the bottom portion of the piston stroke. This type of pulsating spray can cause inconsistent spray application on a customer's skin and is generally undesirable. Additionally, conventional piston-driven pumps often do not include variable speed control and do not allow adequate variation and control of liquid flow-rate.

Application of sunless tanning compositions to human skin using a manual or handheld spray gun generally provides superior quality of application over automated spray booth applications. However, manual hand spraying requires additional workers to assist with spray application. This increases costs and reduces throughput and efficiency for spray tanning salons.

What is needed, then, are improvements in devices and systems for pump-driven sunless spray tanning devices including manual spray systems and automated spray systems.

### BRIEF SUMMARY

The present invention provides a spray tanning system including a liquid reservoir, a spray nozzle, and a peristaltic pump coupled to the reservoir and to the nozzle for delivering spray tanning composition from the reservoir to the nozzle.

In some embodiments, the present invention includes a spray tanning device including a spray nozzle and a peristaltic pump coupled to the spray nozzle. A hose or tube in fluid communication with the spray nozzle is manipulated by the peristaltic pump to deliver sunless spray tanning solution to the spray nozzle.

In some embodiments, the present invention provides a spray tanning booth, or enclosure, having a floor, a ceiling, a wall and a liquid reservoir. The enclosure includes an opening allowing a user to enter and exit the enclosure. A robotic arm spans the opening from the ceiling to the floor. The robotic arm is moveable across the opening. A spray nozzle is disposed on the robotic arm and is oriented toward the wall. The spray nozzle is longitudinally moveable along the robotic arm. A liquid reservoir is disposed on the enclosure. A peristaltic pump includes a pump inlet coupled to the liquid reservoir and a pump outlet coupled to the spray nozzle. In some embodiments, a solenoid valve is disposed between the pump outlet and the spray nozzle.

An object of the present invention is to provide a sunless spray tanning device for applying a spray tanning composition to human skin. The device includes a spray nozzle; a peristaltic pump having a pump outlet coupled to the spray nozzle; and a solenoid valve disposed between the peristaltic pump outlet and the spray nozzle.

A further object of the present invention is to provide a sunless spray tanning device for applying sunless tanning composition to human skin. The device includes a spray tanning enclosure having an opening; a moveable carriage spanning the opening; a spray gun mounted on the carriage; and a first peristaltic pump coupled to the spray gun.

An additional object of the present invention is to provide a handheld HVLP sunless spray tanning device for applying sunless tanning composition to human skin, wherein the device includes at least one peristaltic pump coupled to a spray nozzle.

Numerous other objects, features and advantages of the present invention will be readily apparent to those of skill in the art, upon a reading of the following disclosure, when taken in conjunction with the accompanying drawings.

### BRIEF DESCRIPTION OF THE DRAWINGS

FIG. 1 illustrates a perspective partially cut-away view of an embodiment of a spray tanning device in accordance with the present invention.

FIG. 2 illustrates a side elevation view of an embodiment of a spray tanning device in accordance with the present invention.

FIG. 3 illustrates a left side perspective view of an embodiment of a spray tanning device in accordance with the present invention.

FIG. 4 illustrates a rear elevation view of an embodiment of a spray tanning device in accordance with the present invention.

FIG. 5 illustrates a right side perspective view of an embodiment of a spray tanning device having first and second peristaltic pumps in accordance with the present invention.

FIG. 6 illustrates a detail perspective view of an embodiment of a spray tanning device in accordance with the present invention.

FIG. 7 illustrates a schematic view of an embodiment of a spray tanning device in accordance with the present invention.

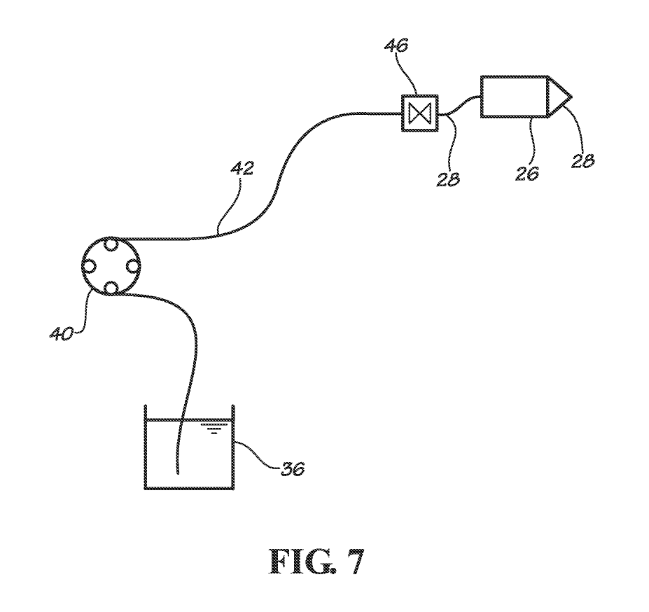

### DETAILED DESCRIPTION

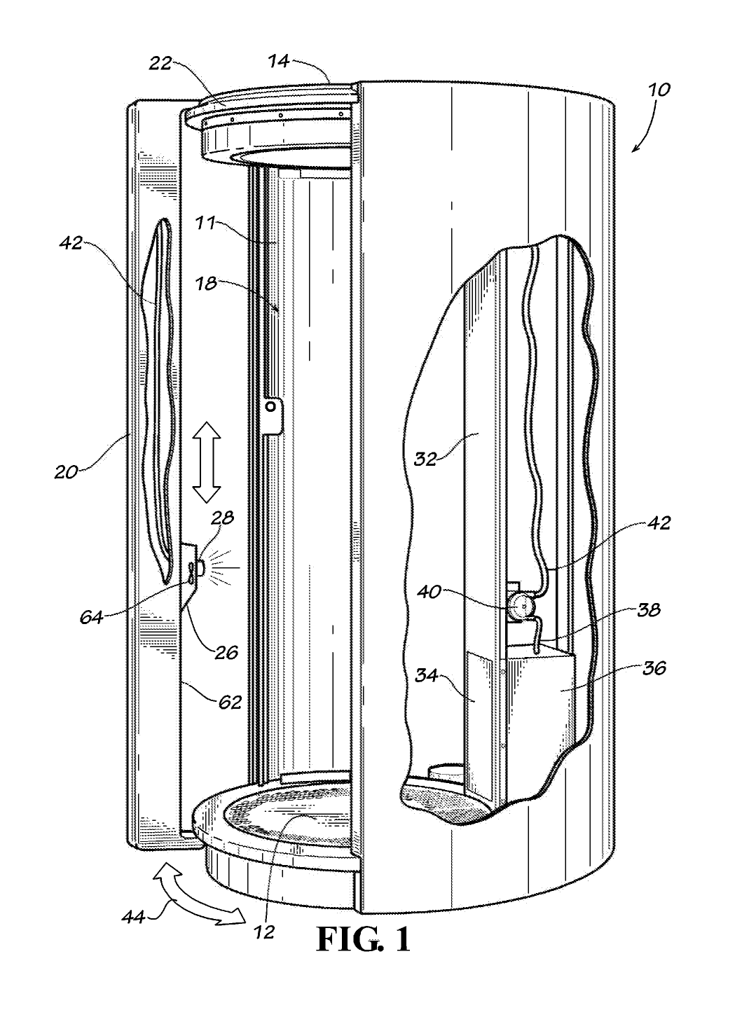

Referring now to the drawings, FIG. 1 illustrates an embodiment of a sunless spray tanning device 10 including a spray booth, or spray tanning booth. The spray tanning booth is generally a free-standing structure forming a partial enclosure 11 having a base, or floor 12, and a top, or ceiling 14. The spray booth, or enclosure 11, is dimensioned such that a user or customer can stand in the enclosure on the floor 12 under the ceiling 14. An opening 18 is generally located on a side of enclosure 11 to allow a customer or user to enter the enclosure 11 and stand on the floor 12. Opening 18 is dimensioned to allow a customer to walk into enclosure 11. During use, a customer or user stands in enclosure 11 on floor 12, and a spray of sunless tanning composition is sprayed onto the customer's skin. The customer may stand at a desired orientation, or may change orientations during a session, to ensure adequate coverage of the skin by the sunless tanning composition.

The present invention generally relates to the mechanism and machinery, and associated methods, for delivering the sunless tanning composition to the user's skin. The present invention includes numerous embodiments as described in the following paragraphs.

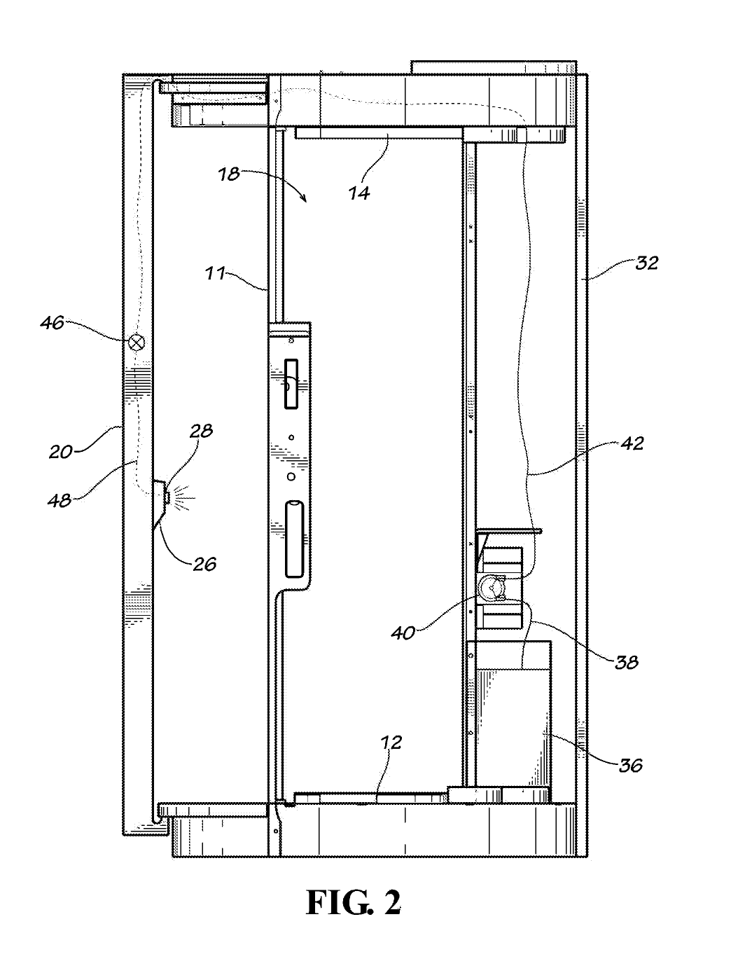

Referring further to FIG. 1, a sunless spray tanning device 10 includes a carriage 20 mounted on the spray booth, or enclosure 11. Carriage 20 generally spans opening 18 in some embodiments. Carriage 20 is illustrated in FIGS. 1 and 2 in a vertical orientation by way of example, but in other embodiments, carriage 20 may include an angled or a horizontal orientation spanning opening 18. Alternatively, carriage 20 does not span completely across opening 18 but rather extends partially across opening 18.

Carriage 20 is moveable relative to the enclosure 11 in some applications. Carriage 20 may be mounted on one or more carriage tracks to allow carriage 20 to be repositioned during a spray-tanning session. A carriage motor or carriage drive is also attached to carriage for controlling the movement of the carriage 20. As seen in FIG. 1, carriage 20 is bi-directionally moveable laterally along a portion of the outer perimeter of opening 18 on enclosure 11 as indicated by arrow 44. In some embodiments, a carriage track 22 is disposed on enclosure 11. Carriage 20 is configured to move on carriage track 22. Carriage track 22 may include an upper carriage track rail and a lower carriage track rail in some embodiments.

As seen in FIG. 1 and FIG. 2, a spray gun 26 is mounted on carriage 20. Spray gun 26 can include any suitable spray gun known in the art for emitting sunless spray tanning compositions, such as a high volume low pressure (HVLP) spray gun. Spray gun 26 generally includes at least one spray nozzle 28 from which a spray of sunless tanning composition is emitted toward a user's skin when the user is standing inside the enclosure 11. In various applications, a spray of sunless tanning composition may be emitted from spray gun 26 while carriage 20 is moving along carriage track 22.

Additionally, in some embodiments, spray gun 26 is longitudinally moveable along a spray gun track 62 on carriage 20. For example, as seen in FIG. 1 and FIG. 2, spray gun 26 may move up or down along spray gun track 62. During use, spray gun 26 is operable to emit a spray of sunless tanning composition while simultaneously translating along spray gun track 62 on carriage 20. Also, during said movement of spray gun 26, carriage 20 may simultaneously move along carriage track 22 relative to enclosure 11. As such, spray gun 26 and spray nozzle 28 may move in at least two dimensions during a spray tanning session. This allows the emission location of the spray to be repositioned in real time during the spray tanning session, thereby allowing the user to remain stationary while the spray gun 26 is moved to cover all areas of the user's skin. In some applications, this automated movement of spray gun 26 simulates a manual spray tanning session.

As seen in FIG. 1 and FIG. 2, in some embodiments, sunless spray tanning device 10 includes a tower 32, or cabinet, extending upwardly from floor 12, or base. Tower 32 provides an internal housing for various hardware components on the device. Such components may include one or more blower fans, ducting, pumps, reservoirs, wiring, motors, etc. Tower 32 may extend completely from floor 12 to ceiling 14, or alternatively may extend only partially from floor 12 toward ceiling 14. Tower 32 includes a rectangular cross-sectional profile in some embodiments, but may also include other suitable shapes in various other embodiments not shown.

Liquid reservoir 36 is disposed on the enclosure 11 in some embodiments. In alternative embodiments, liquid reservoir 36 may be located remote from device 10 and may be coupled to device 10 via one or more connection hoses. Liquid reservoir 36 includes one or more containers for storing liquid sunless tanning composition.

A peristaltic pump 40, or first peristaltic pump in embodiments having more than one pump, is disposed on or otherwise attached to the device 10. In some embodiments, peristaltic pump 40 is mounted on sunless spray tanning device 10 at a location inside the enclosure 11, and preferably inside tower 32. In some embodiments, peristaltic pump 40 is located near the liquid reservoir 36, as seen in FIG. 1. In alternative embodiments, peristaltic pump 40 is located remote from first liquid reservoir 36 at a location such as on or inside carriage 20.

Peristaltic pump 40 may include any suitable peristaltic pump known in the art for pumping fluids. A peristaltic pump is generally defined as a pump that passes one or more surfaces over a compressible tube to force fluid through the tube. The surfaces may include rigid surfaces or rollers, and the surfaces generally move in a rotary manner. The rollers may be powered by a pump motor or by any other suitable drive mechanism. In some embodiments, peristaltic pump 40 includes a conventional peristaltic pump such as a WP100 series pump manufactured by Welco Co., Ltd. of Tokyo, Japan. For example, in some embodiments, peristaltic pump 40 includes a Welco pump model WP10P1 including a 1.6 mm inner diameter Pharmed BPT internal tube operable at up to about 145 psi and at a variable speed of about 55-225 rpm. In a preferred embodiment, peristaltic pump 40 includes a variable 8-24VDC brush motor and at least four rollers configured for compressing and rolling against the tubing for moving fluid through the tubing. In some embodiments, peristaltic pump 40 is configured to provide a liquid flowrate of about 11 ml of sunless tanning composition through the pump per minute.

Peristaltic pump 40 is generally chosen to be able to deliver liquid sunless tanning compositions having widely-ranging viscosities to spray gun 26. In some embodiments, the liquids to be delivered via peristaltic pump 40 include a sunless tanning composition having a viscosity between about 1 centipoise to about 15 centipoises. However, in various other applications, the devices and methods of the present invention are operable to deliver sunless tanning compositions having even greater or lower viscosities.

Referring further to FIG. 2, in some embodiments, peristaltic pump 40 is mounted inside tower 32 in enclosure 11. Peristaltic pump 40 includes a pump outlet coupled to spray nozzle 28 on spray gun 26 via a pump outlet hose 42. In some embodiments, pump outlet hose 42 includes a tube extending away from pump 40 upwardly toward ceiling 14. Pump outlet hose 42 may be routed through ceiling 14 and across a sliding joint across carriage track 22 into carriage 20. Pump outlet hose 42 then travels downwardly through carriage 20 to a spray head, or spray gun 26 in some embodiments. The pump outlet on peristaltic pump 40 is coupled to spray nozzle 28 on spray gun 26 as long as a liquid communication path is established between the hose mechanically operated on by the pump and the spray nozzle. Pump outlet hose 42 may be subdivided into multiple hose segments being joined together by one or more hose connectors in some embodiments.

Spray gun 26 includes at least one spray nozzle 28 from which a spray of sunless tanning composition and pressurized gas are ejected toward the interior of enclosure 11. In some applications, spray gun 26 includes a high volume, low pressure (HVLP) spray gun. In such embodiments, a pressurized gas supply hose is also coupled to spray gun 26 and/or spray nozzle 28. In some embodiments, the pressurized gas supply hose is coupled at the opposite end to a compressed air source such as a blower, a compressed gas storage tank, or a compressor. HVLP spray guns are a unique type of spray gun known in the art for sunless tanning composition application. Some HVLP spray guns typically require both a pressurized gas supply line and a liquid tanning composition supply line. Alternatively, in some embodiments, spray gun 26 includes an internal turbine 64 for providing a flow of air through spray nozzle 28 and for emitting sunless tanning composition without requiring an additional pressurized gas line coupled to spray gun 26.

Referring further to FIG. 2, a valve 46 is positioned between pump 40 and spray nozzle 26. Valve 46 in some embodiments is an electronically controlled solenoid valve. Valve 46 is disposed on carriage 20 between spray gun 26 and pump outlet supply line 42 in some embodiments. Valve 46 may include an electronically-controlled three-way valve that can be selectively opened and closed during operation of the device. Additionally, a metering valve may also be disposed on or near the outlet of valve 46 in some embodiments. The metering valve may establish a back-pressure in pump outlet hose 42 even when solenoid valve 46 is open.

A charge line 48 is disposed between solenoid valve 46 and spray gun 26 in some embodiments. Charge line 48 includes a section of tubing or hose further coupling the output of solenoid valve 46 to the tanning composition input on spray gun 26. During use, a pressurized volume of sunless tanning composition to be applied to human skin travels through charge line 48 to spray nozzle 28 for ejection. A stream of compressed gas or air from turbine 64 may be introduced to a liquid stream of sunless tanning composition at or near spray nozzle 28. The interaction of the gas with the liquid sunless tanning composition forcefully ejects the sunless tanning composition from the spray nozzle 28 and also breaks the liquid stream of sunless tanning composition into a spray of droplets. In some embodiments the peristaltic pump outlet is coupled to the spray nozzle 28 via both pump outlet hose 42 and charge line 48, including a valve 46 positioned between the pump outlet hose 42 and the charge line 48.

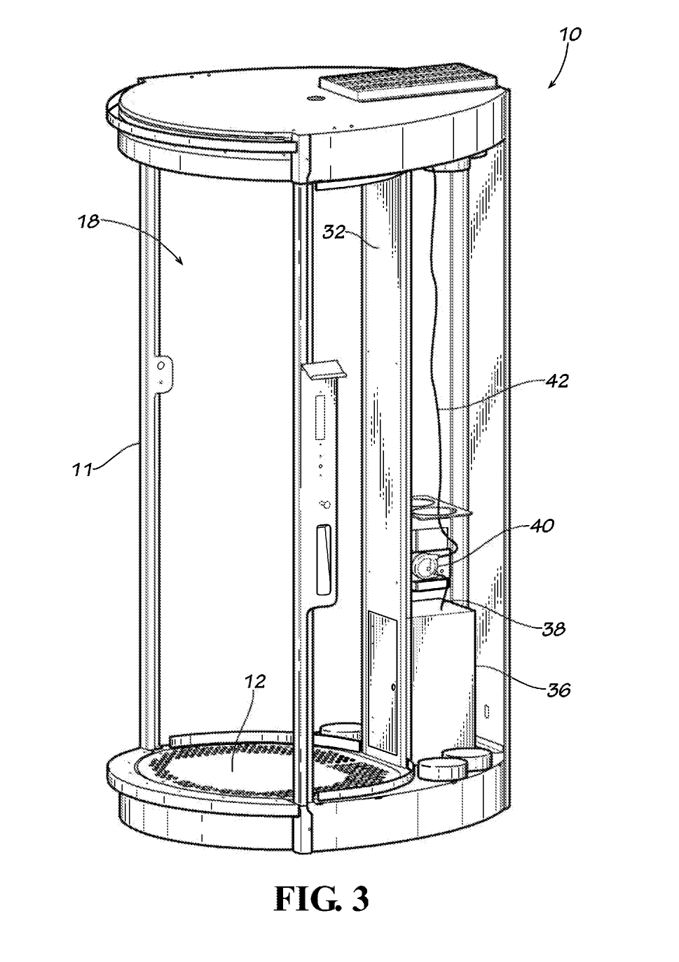

Referring further to FIGS. 3-6, in some embodiments, it may be desirable to provide a spray device 10 having the ability to selectively spray multiple different tanning spray compositions without switching out the liquid reservoir. For example, a first customer may desire a lighter spray tan composition, and a second subsequent consumer may prefer a darker spray tan composition. In many applications, it would generally be advantageous to have the ability to provide different sprays of sunless tanning composition without having to manually change out the liquid reservoir between spray tanning sessions. The present invention in some embodiments addresses this problem by providing a sunless spray tanning device 10 capable of selectively alternating between multiple liquid reservoirs that contain different sunless tanning compositions.

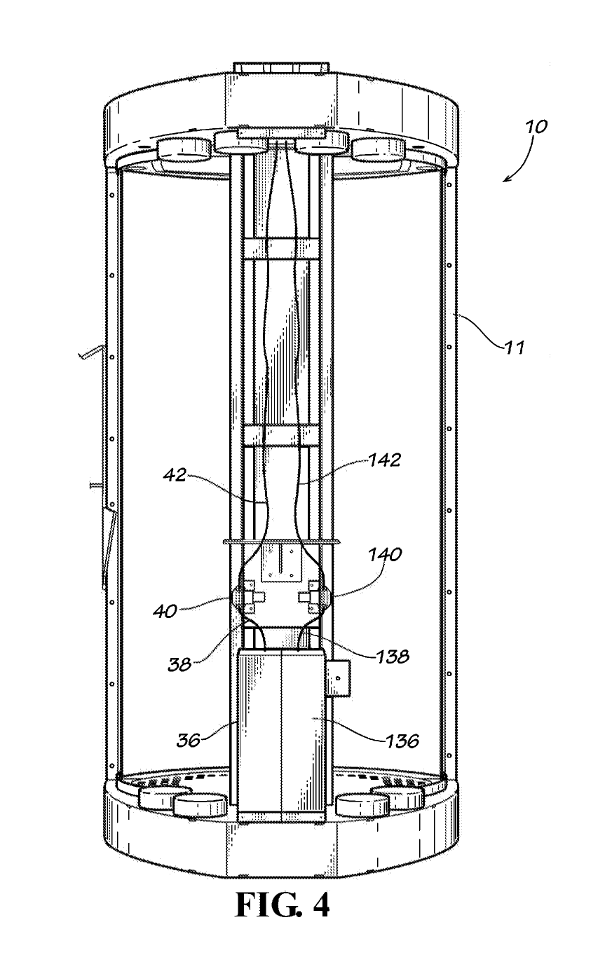

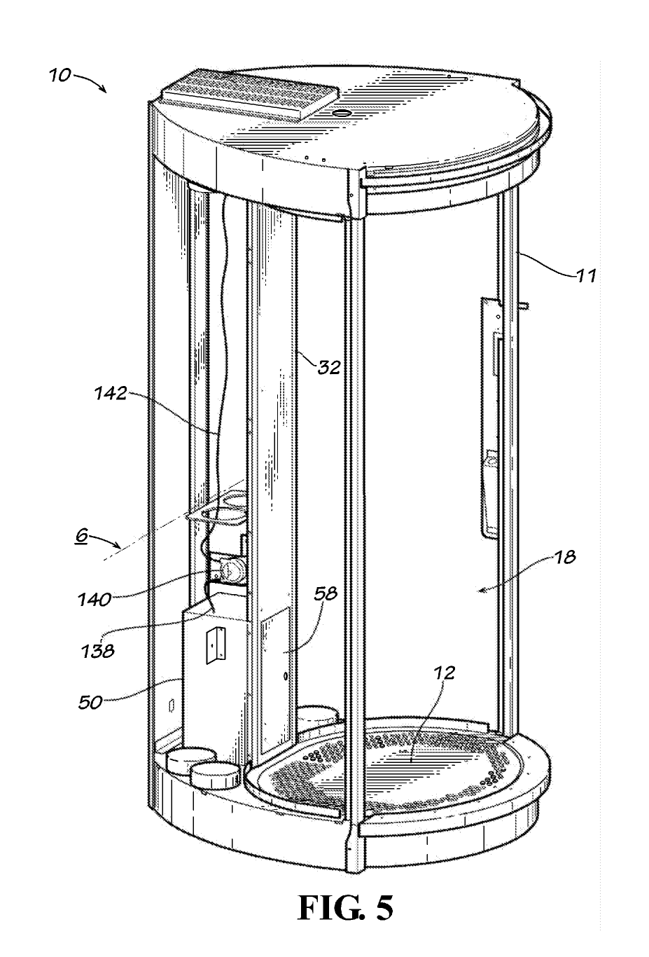

In some applications, as seen in FIG. 4, the present invention provides a sunless spray tanning device 10 having first and second peristaltic pumps 40, 140 coupled to independent reservoirs containing different sunless tanning compositions. First peristaltic pump 40 is coupled to a first liquid reservoir 36 containing a volume of a first sunless tanning composition, and second peristaltic pump 140 is coupled to a second liquid reservoir 136 containing a volume of a second sunless tanning composition. First and second liquid reservoirs 36, 136 may both be housed in a chamber or cabinet 50 in tower 32 below first and second peristaltic pumps 40, 140, as seen in FIG. 5. A user or device operator may select a preferred sunless tanning composition for application during a sunless tanning session in the enclosure 11. In additional embodiments, more than two peristaltic pumps and corresponding liquid reservoirs may be included on device 10 to provide more than two options of sunless tanning composition.

Second peristaltic pump 140 includes a second pump inlet hose 138 extending from the second reservoir 136 to the second pump inlet. A second pump outlet hose 142 extends from the second pump outlet to the spray gun 26 in some embodiments. Second pump outlet hose 142 generally delivers a second sunless tanning composition to spray gun 26 from second reservoir 136. Second pump outlet hose 142 may be coupled directly to spray gun 26 in some embodiments. Second peristaltic pump includes a second pump outlet coupled to spray nozzle 28 on spray gun 26 via second pump outlet hose 142 in some embodiments.

In some applications, first solenoid valve 46 is a three-way valve, and both first pump outlet hose 42 and second pump outlet hose 142 are coupled to first solenoid valve 46. As such, first solenoid valve 46 may control whether liquid is supplied to spray gun 26 from first pump outlet hose 42 or from second pump outlet hose 142.

Alternatively, in some embodiments, a second solenoid valve is disposed between second pump outlet hose 142 and spray gun 26. The second solenoid valve is similar to the first solenoid valve 46 described herein. A second charge line may also extend between the outlet from the second solenoid valve and the spray gun 26.

In further embodiments, second pump outlet hose 142 is coupled to a second spray gun mounted on carriage 20 having a second spray nozzle such that the different sunless tanning compositions can be emitted from separate nozzles.

As seen in FIG. 4 and FIG. 5, first and second liquid reservoirs 36, 136 may be housed in a cabinet 50 on tower 32. A door 58 is disposed on cabinet 50 in some embodiments. Door 58 generally faces inwardly toward the interior of enclosure 11 such that door 58 may be accessed from the interior of enclosure 11. As such, an operator may directly change, replace or modify first and second liquid reservoirs 36, 136 from the interior of enclosure 11 without being forced to move around to the backside of the device. Cabinet 50 also provides protection for first and second liquid reservoirs 36, 136, which may include plastic containers or bags containing liquids.

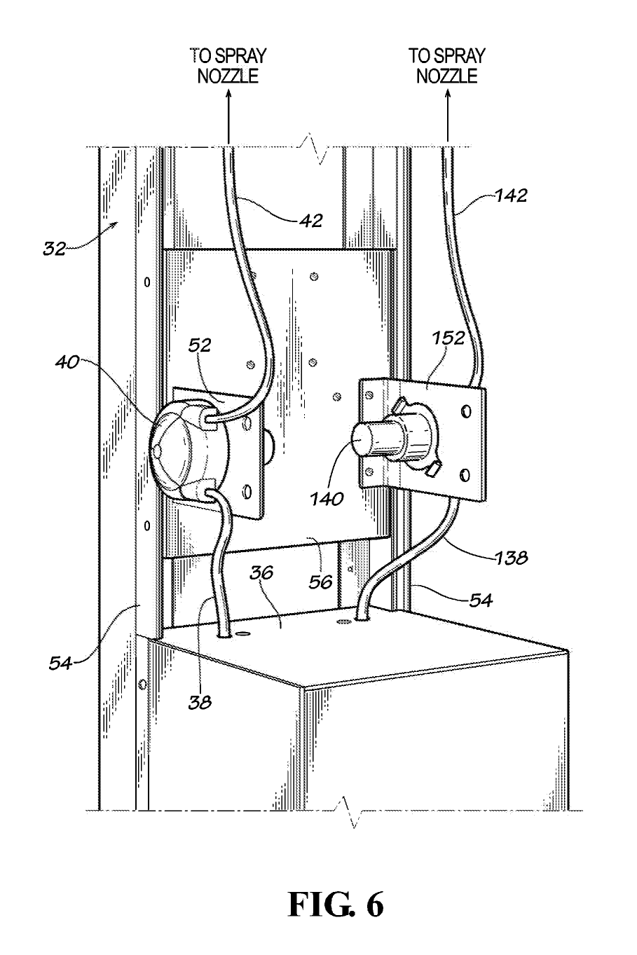

Referring further to FIG. 6, each peristaltic pump may be mounted on a separate mounting bracket housed within tower 32. For example, first peristaltic pump 40 is mounted on first pump bracket 52. First pump bracket 52 includes an L-shaped bracket with an opening for allowing passage of the pump motor. Similarly, second peristaltic pump 140 is mounted on a second pump bracket 152 positioned opposite first pump bracket 52. Second pump bracket 152 also includes an L-shaped bracket in some embodiments having an opening for passage of the motor on second peristaltic pump 140. First and second pump brackets 52, 152 are mounted on a mounting plate 56 along frame 54 of tower 32 in some embodiments. It is understood that numerous other mounting configurations for first and second peristaltic pumps 40, 140 may be used.

Although the various embodiments in the figures illustrate the first and second pump outlet hoses 42, 142 extending upwardly through tower 32, through ceiling 14 and downwardly into carriage 20 toward spray gun 26; in other embodiments the pump outlet hoses may be routed downwardly through tower 32, through the floor 12 and upwardly into carriage 20 to spray gun 26. Such alternative embodiments are clearly contemplated within the scope of the present invention.

In further embodiments, the present invention provides a method of generating a spray of sunless tanning composition for application to human skin. The method includes the steps of: (a) providing a spray gun having a spray nozzle, the spray nozzle coupled to the outlet of a peristaltic pump, the peristaltic pump inlet being coupled to a liquid reservoir of sunless tanning composition; (b) advancing a volume of sunless tanning composition through the peristaltic pump toward the spray nozzle; and (c) emitting the sunless tanning composition from the spray nozzle. In some embodiments, the method further includes the step of restricting the flow of sunless tanning composition to the spray nozzle using a solenoid valve disposed between the peristaltic pump and the spray nozzle.

Additionally, in some embodiments, the present invention provides a sunless spray tanning device including a peristaltic pump having a pump outlet coupled to a spray nozzle, wherein the spray nozzle is located on a handheld spray gun. In some embodiments, the handheld spray gun is a manual HVLP spray gun.

Thus, although there have been described particular embodiments of the present invention of a new and useful Sunless Spray Tanning Devices and Methods, it is not intended that such references be construed as limitations upon the scope of this invention except as set forth in the following claims.

What is claimed is:

1. A sunless spray tanning device for applying a spray tanning composition to human skin, comprising:
a spray nozzle;
a peristaltic pump having a pump outlet; and
an electronic solenoid valve disposed between the pump outlet and the spray nozzle, wherein the pump outlet is in fluid communication with the spray nozzle.

2. The device of claim 1, wherein the pump outlet is a tube.

3. The device of claim 1, wherein the solenoid valve is a three-way solenoid valve.

4. The device of claim 1, further comprising:
a spray booth; and
a moveable carriage mounted on the spray booth,
wherein the spray nozzle is disposed on the moveable carriage.

5. The device of claim 4, wherein the spray nozzle is longitudinally moveable along the carriage.

6. The device of claim 5, further comprising:
the peristaltic pump including a pump inlet; and
a liquid reservoir coupled to the pump inlet.

7. The device of claim 6, further comprising an opening defined in the spray booth, wherein the carriage spans the opening.

8. The device of claim 1, further comprising a stationary spray booth, wherein the spray nozzle is mounted on the spray booth.

9. The device of claim 1, further comprising a handheld spray gun, wherein the spray nozzle is mounted on the spray gun.

10. The device of claim 1, further comprising a second peristaltic pump having a second pump outlet in fluid communication with the spray nozzle.

11. A sunless spray tanning device for applying sunless tanning composition to human skin, comprising:
a spray tanning enclosure having an opening;
a moveable carriage spanning the opening;
a spray gun mounted on the carriage; and
a first peristaltic pump having a first pump outlet in fluid communication with the spray gun.

12. The device of claim 11, wherein the spray gun is longitudinally moveable along the carriage.

13. The device of claim 12, further comprising a second peristaltic pump having a second pump outlet in fluid communication with the spray gun.

14. The device of claim 13, further comprising a solenoid valve disposed between the first peristaltic pump and the spray gun.

15. The device of claim 13, further comprising a first liquid reservoir coupled to the first peristaltic pump, the first liquid reservoir positioned in the enclosure.

16. The device of claim 15, further comprising a second liquid reservoir coupled to the second peristaltic pump, the second liquid reservoir positioned in the enclosure.

17. The device of claim 16, further comprising:
the enclosure including a floor and a ceiling; and
a tower extending from the floor to the ceiling in the enclosure,
wherein the first and second liquid reservoirs are disposed in the tower.

18. The device of claim 17, wherein the first and second peristaltic pumps are disposed in the tower.
